(19) United States
(12) Patent Application Publication
(10) Pub. No.: US 2017/0105665 A1
(43) Pub. Date: Apr. 20, 2017

(54) VEIN ACCESS NEEDLE GUIDE ASSEMBLY AND METHODS OF USE FOR VENIPUNCTURE AND VEIN STABILIZATION

(71) Applicant: V-ALIGN, INC., Dobbs Ferry, NY (US)

(72) Inventors: Sundaram Ravikumar, Briarcliff Manor, NY (US); Vikram Ravikumar, Briarcliff Manor, NY (US); Guy Osborne, Trumbull, CT (US); Harry Allan Alward, Shelton, CT (US)

(73) Assignee: V-ALIGN, INC., Dobbs Ferry, NY (US)

(21) Appl. No.: 14/886,765

(22) Filed: Oct. 19, 2015

(51) Int. Cl. A61B 5/15; A61F 13/02

(52) U.S. Cl. CPC A61B 5/150732; A61B 5/15003; A61B 5/150274; A61B 5/150305; A61B 5/150503; A61B 5/15074; A61F 13/0269

(57) ABSTRACT

A needle guide assembly for vein access including (a) a guide having a convex portion, a concave portion, an aperture and a stabilization means for stabilizing a vein against moving away and escape puncture, where the concave portion and the convex portion meet each other to define the aperture between the concave portion and the convex portion, each of the convex portion and the concave portion extending away from the aperture in opposite directions from each other in a tapering manner and terminating the tapering at respective locations away from the peripheral edge, the convex portion being made of a transparent or translucent material, the stabilization means being a pair of elongated indentations that extend substantially parallel to each other and between which are the convex portion, the concave portion and the aperture, and (b) a retaining means for retaining the guide to skin. A venipuncture method is includes retaining the needle guide assembly onto the skin of a patient and using the needle guide assembly to guide a needle to puncture the skin and access a vein.

### RELATED APPLICATIONS

This application claims priority from provisional application Ser. No. 61/648,585 filed Oct. 7, 2013 and PCT Application No. PCT/US14/059576 filed on Oct. 7, 2014 and Ser. No. 14/652,243 filed Jun. 15 2015.

### FIELD OF THE INVENTION

The present invention relates to medical products and methods of their use, and more particularly to a needle guide assembly and method of venipuncture, is through which needles can be easily and accurately placed into a patient's vein with improved vein stabilization.

### BACKGROUND OF THE INVENTION

Intravenous access is a ubiquitous component of some of the most common medical treatments and procedures. Most common access is a percutaneous injection via piercing the skin with a hollow needle inserted into a vein, whether the vein is in the person's hand, arm, groin, neck or other body part. Venipuncture to obtain a blood sample is most commonly obtained from the median cubital vein, which lies within the cubital fossa anterior to the elbow, as this vein lies close to the surface of the skin so it is easily accessible while not surrounded by many nerves so as to minimize the pain for the individual.

Phlebotomy via venipuncture occurs hundreds of millions of time per year worldwide, if not a billion. Accessing a vein is critical, because veins are the conduit through which medical professionals draw blood from as well as inject fluids into a patient's circulatory system.

Most common reasons for venipuncture is to obtain blood for diagnostic purposes, to monitor levels of blood components, and to administer therapeutic treatments such as medications (e.g., intravenous antibiotics), nutrition, or chemotherapy for cancer patients. Other reasons for venipuncture include removing blood due to excess levels of iron (e.g., chelation therapy) or erythrocytes (i.e., red blood cells) or to collect blood for later uses such as donor blood and transfusions. All of these processes require access to a vein via venipuncture.

During conventional venipuncture the user (e.g., phlebotomist, nurse, doctor or other medical personnel) positions the needle to be inserted over the chosen vein at an angle so as to make sure the needle does not puncture the vein and exit the vein on the other side, thus not being in contact with the blood supply within the vein. The user's goal is to position the needle tip within the vein so that there is access to the blood within the vein, whether for blood collection or for insertion of medication, fluids or other compositions into the blood supply of the individual or patient. However, this goal may be complicated as the vein chosen for the venipuncture is typically supported by fatty tissue in the cells that make up the connective tissue, and the chosen vein may lie between the skin and the underlying muscle or bone. So typically the user elies upon visual cues to locate the vein and perform the venipuncture.

Further, the user may position a tourniquet at a location such that the blood increases below the tourniquet and the vein “throbs” making it easier to locate and puncture. The user may also touch and feel the skin of the individual or patient and attempt, to locate the vein in this manner, together with a visual observation.

Once the needle tip is inserted into the skin the user may also use tactile senses by trying to “feel” where the needle tip is in relation to the vein. Since the needle tip is inserted into the skin layer, it passes through the fatty tissue layer and into the wall of the vein which acts as a third layer. Depending on the resistance felt by the user, the needle angle may be adjusted as well in order to puncture the vein wall without passing through to the other side, of the vein and ensuring contact with the blood within the vein. Once the needle tip is successfully within the vein and in contact with the blood the needle angle may be adjusted depending on the connection at the other end of the needle such as a vacuum bottle for blood collection or an intravenous liquid for insertion into the blood flow of the individual or patient.

In certain instances the individual patient may experience anxiety or even pain depending on the needle tip diameter, the structure of the individual's veins and whether his or her veins have a tendency to, collapse when punctured, and the experience level of the user performing the venipuncture. If the venipuncture is not successful there may be repeated attempts until successful completion. Multiple attempts may add to the pain and the anxiety of the individual or patient, especially for a pediatric patient, elderly patient, a special needs individual, or a person with anxiety issues.

One problem which may occur during the venipuncture is movement of the vein targeted for puncture. As the vein is often difficult to hold steady the vein itself being targeted for puncture may roll or move sideways. Further, the needle tip itself on insertion into the skin layer may in fact push the vein to the side and out of the path of the needle tip itself. The patient may also move the body part where the venipuncture is being performed. Upon any of these problems the venipuncture is not successful and further attempts must be made resulting in certain trauma to the individual or patient's skin layer and layers below, as well as to the individual's psyche and anxiety.

In certain circumstances the user may try to use his or her own finger applying pressure to the patient's body part so as to secure the vein from movement, but then the user is exposed for any subsequent sudden movement by the patient and possible puncture of the needle into the user's finger rather than puncturing the vein.

Vein access is critically important especially in patients who need continual access to their circulatory system, such as patients receiving chemotherapy or dialysis. Renal failure is a prevalent chronic disease in the United States with approximately 600,000 patients. The term renal failure refers to the inability of the patient's kidneys to properly rid the patient's blood supply of waste products such as creatinine, urea, and free water. The most common treatment for this condition is hemodialysis. Hemodialysis (or commonly referred to as dialysis) is the process of extra-corporally removing the waste product from the blood supply by circulating the patient's blood through a dialysis machine, which “purifies” the blood of unwanted waste materials and returns the blood to the patient.

There are various techniques in which dialysis is done, but the more desirable and common technique is through what is known as an AV fistula. For the purposes of dialysis, an AV for Arterial Venous) fistula is a surgical procedure to connect the vein and artery. The AV fistula is accessed by inserting two needles, one needle for drawing the blood to circulate through the dialysis machine and a second needle to return to blood back into the patient.

One common complication with this procedure occurs when the vein is not fully dilated—the needle often does not puncture the vein properly and causes blood to extravasate, causing the swelling and hematoma formation. Worse, improperly puncturing the AV fistula can actually damage the AV fistula making recannulation not possible. Apart from the destruction of the fistula, the patient suffers from pain and discomfort especially from the hematoma. Ideally, the needle should enter the vein directly only piercing the vein where it entered. By entering directly into the vein and not puncturing the side or back walls of the vein, the needle can access the AV fistula and allow the dialysis to commence with minimal extravasation and the morbidities associated with it.

Further problems occur with the angle of insertion of the needle into the vein. The needle must be inserted at a certain angle so as to puncture the vein but not the lower wall of the vein while the needle remains at an optimum angle so that the blood can flow freely into the collection vehicle and quickly to have the venipuncture procedure successfully complete. The needle must also be secured during the venipuncture so that the needle angle does not vary during the procedure affecting the collection and possibly incurring pain and injury to the patient.

Thus, there exists a need for a guide to assist in the proper insertion of a needle into a vein. While certain vein guides have been the subject of patents and applications, they have notable problems. For instance, they may be too binding and constricting on the patient, especially an elderly, pediatric or anxious patient. They may also be too large and wieldy to use easily in a clinic or blood collection site such as Quest Diagnostics, Inc. or Laboratory Corporation of America (LabCorp).

A further need exists for a vein stabilizer for use during venipuncture. There is a continuing need for a vein guide which assists in the angle of insertion of the needle into the target vein.

A further need exists for a vein guide which also improves the stabilization of the target vein so that the vein does not roll during the venipuncture procedure.

Yet a further need exists for a cheaper and easily available guide which may be used by hospitals, clinics and even small individual doctor's offices. A further need exists for a guide which is flexible and not constricting on a patient while in use. A further need exists for a guide which secures the needle once inserted into the individual's body part.

These and other needs are met by the present invention including a I.5 needle guide assembly apparatus for vein access and method of use. Other advantages of the present invention will become apparent from the following description and appended claims.

### BRIEF DESCRIPTION OF THE DRAWINGS

FIG. 1 is one embodiment of a needle guide assembly for vein access of the present invention.

FIG. 2 is one embodiment of a guide slide assembly of the present invention.

FIG. 3 is an exploded view of one embodiment of the present invention.

FIG. 4 is a further embodiment of the present invention.

FIG. 5 is an embodiment of the present invention and a needle ready for use with the present invention.

FIG. 6 is an embodiment of the present invention in use on an arm of a patient and showing a needle in use with the present invention.

FIG. 7 is another embodiment of the present invention including a pair of stabilization means.

FIG. 8 is a side view of the embodiment of the present invention of FIG. 7.

FIG. 9 is another embodiment of the present invention including a pair of stabilization means and an angled support.

FIG. 10 is another embodiment of the present invention including a is pair of stabilization means and a securing tab showing the needle inserted within the present invention.

FIG. 11 is another embodiment of the present invention including a pair of stabilization means and an angled support and a securing tab with a needle hub having a locking means.

FIG. 12 is another embodiment of the present invention including a pair of stabilization means and an angled support and a securing tab with a needle hub having a locking means.

FIG. 13 is a zoomed view of the present invention of FIG. 12.

FIG. 14 is another embodiment of the present invention including a pair of stabilization means and an angled support and a securing tab and a locking means for connection to a needle hub.

### SUMMARY OF THE INVENTION

This invention provides an apparatus for guiding a needle into a vein. The invention further provides an apparatus for securing an inserted needle into a vein during venipuncture. The invention further provides for a method of use including venipuncture.

The invention includes a needle guide assembly for vein access. The vein access needle guide assembly is the conduit through which the needle is guided, inserted and may be secured during venipuncture. A portion of the vein access needle guide assembly may be transparent or translucent to allow the user performing the venipuncture to properly align the needle with the target vein in n individual or patient. In one embodiment, the vein access needle guide assembly apparatus includes a retaining means such as an adhesive backed plastic piece with an aperture such as a hole, functioning as a guide, in the middle of the plastic piece. In this embodiment the aperture may have a “track” that when aligned properly above the targeted vein or even a fistula, would guide the needle into the vein at the proper angle, therefore reducing the risk of extravasation. In this embodiment the retaining means further includes a pair of stabilizing means and may optionally include an angled, support in line with the aperture to support the needle when inserted as well as an optional tab or locking means to secure the vein access needle guide assembly to the inserted needle. The vein access needle guide assembly may include additional portions joined on each side of a guide slide assembly to secure the vein access needle guide assembly to the patient's skin when in use, which have an adhesive backing or other configuration to be affixed onto the skin.

The method of use of the invention includes placing the inventive vein access needle guide assembly onto the body part of the individual or patient at the location of the target vein or fistula, retaining the vein access needle guide assembly on the skin such as by adhesion, inserting the needle into the groove and aperture of the vein access needle guide assembly, puncturing the vein while at the same time securing the needle in place within the vein while extending out of the skin. Optionally if a locking means is included in the inventive vein access needle guide assembly the user will lock the inserted needle to the locking means.

Other embodiments of the inventive apparatus and method are disclosed below.

### DETAILED DESCRIPTION OF THE INVENTION

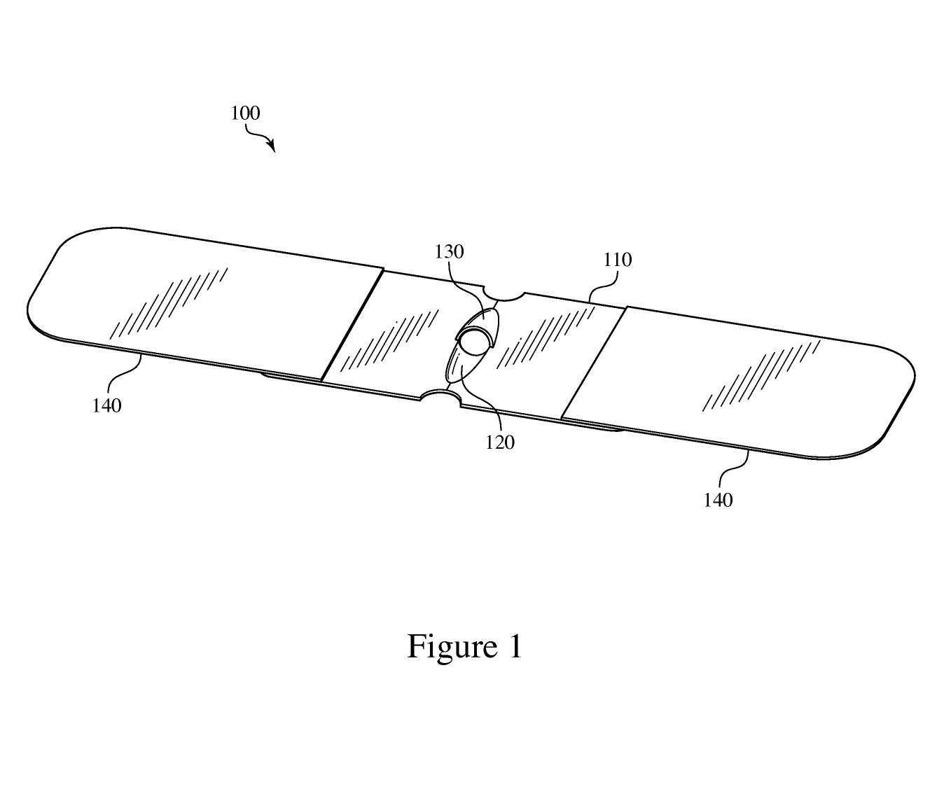

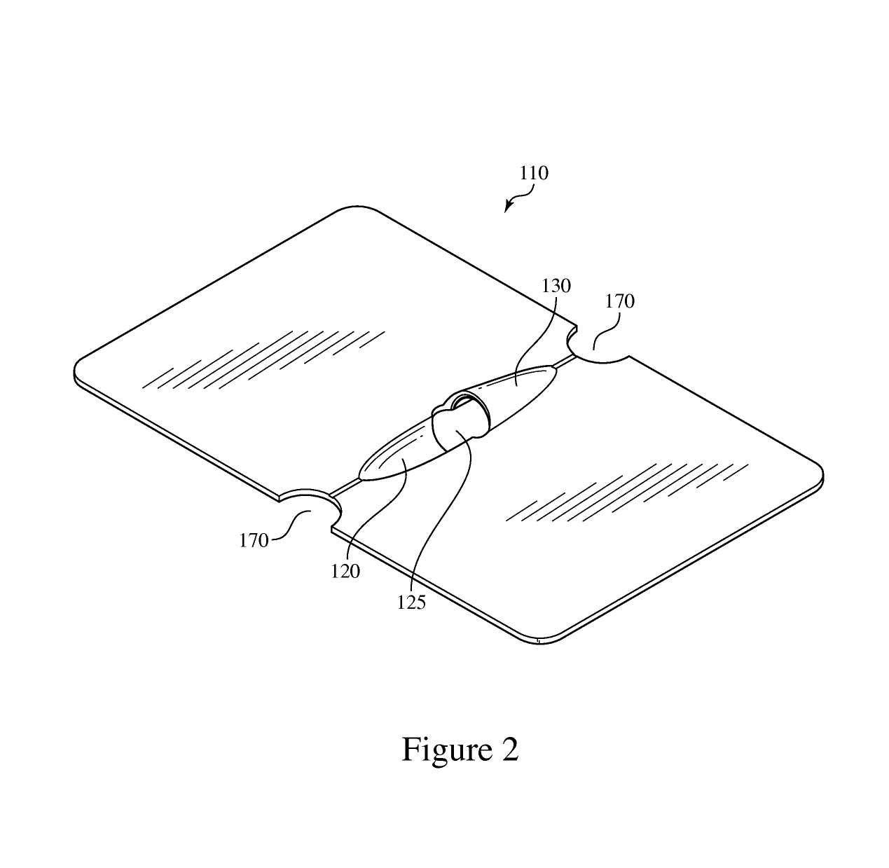

Reference will now be made to the drawings wherein like reference numerals may identify similar structural features or aspects of the subject invention. For purposes of explanation and illustration, and not limitation, a view of an exemplary embodiment of the needle guide assembly for vein access is shown in FIG. 1 and is designated generally by reference character 100. Other embodiments of the vein access needle guide assembly and methods in accordance with the invention, or aspects thereof, are provided in FIGS. 2 through 14.

Referring now to FIGS. 1 through 6, a vein access needle guide assembly 100 is shown to have a guide slide assembly 110 including a guide slide comprising a concave portion 120 and a convex portion 130. The guide slide assembly 110 may be made out of a rigid or semi-rigid material and could be translucent, or transparent. The guide slide assembly 110 has convex 130 and concave 120 portions to accommodate and guide a needle as it is inserted into the patient. Further, the convex portion 130 of the guide slide assembly 110 may secure the needle as it will overlay the needle when inserted into the patient's skin during the venipuncture. In certain embodiments the convex and concave portions may be reversed, or only one portion may be included, either the convex portion alone or the concave portion alone.

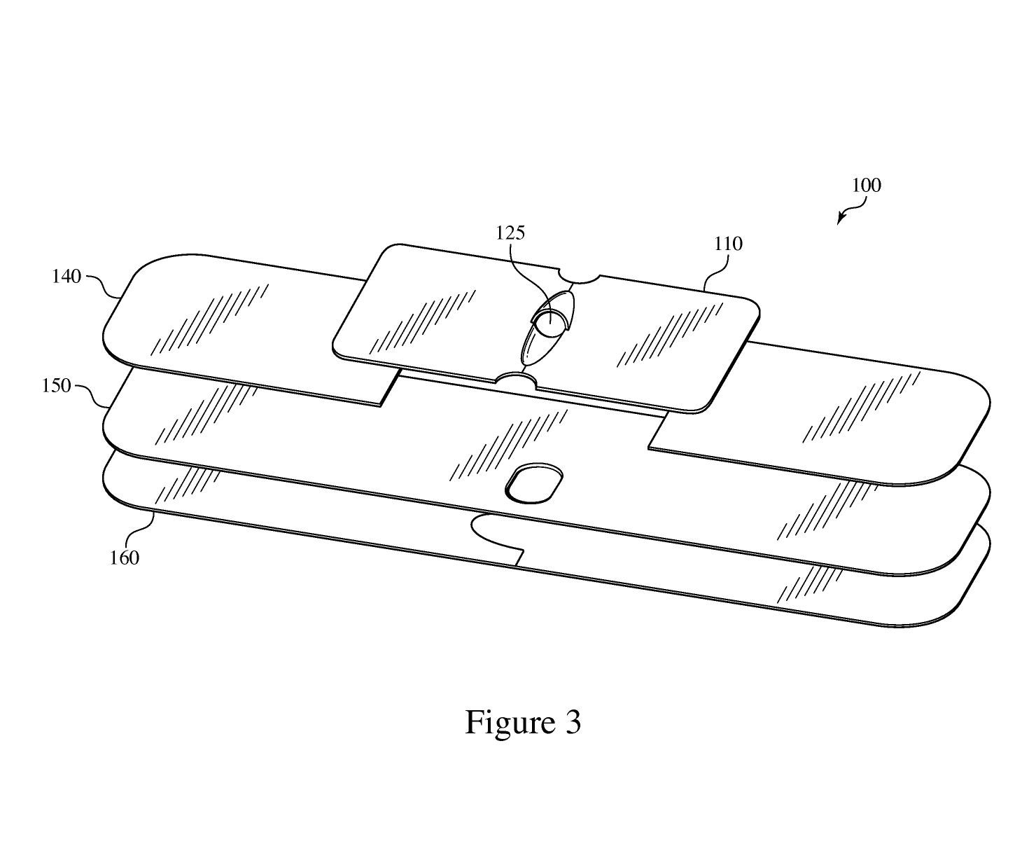

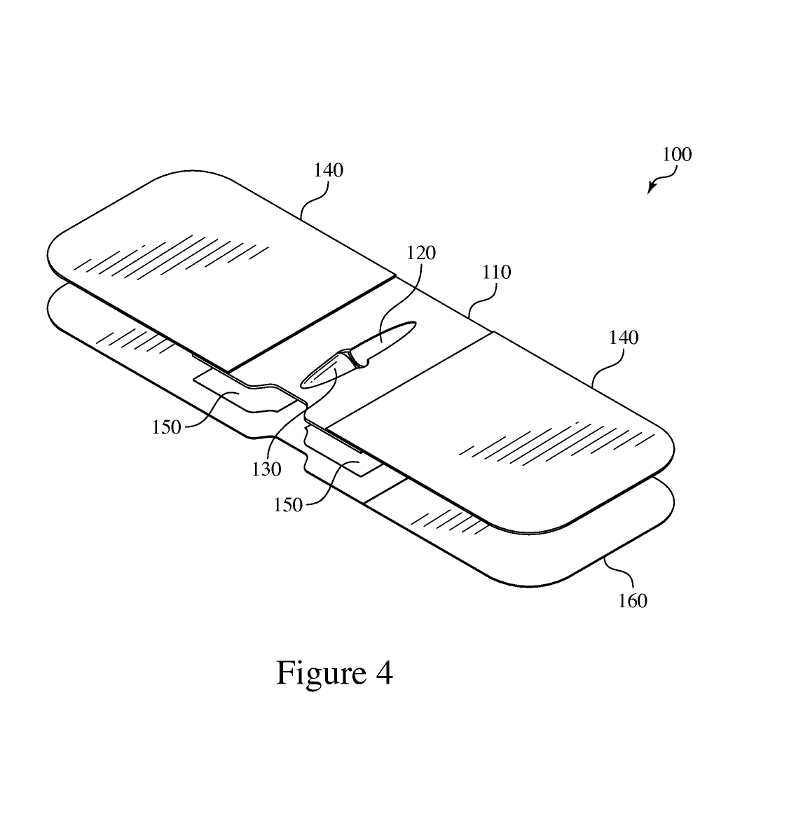

The vein access needle guide assembly 100 further includes a retaining means such as at least one adhesive side strap 140 which secures the vein access needle guide assembly 100 to the skin of the patient when in use. The adhesive side strap 140 may be comprised of a polymer film, plastic, nonwoven or woven material, or is the material may be combinations thereof. This material is preferably non-absorbent or essentially non-absorbent and preferably flexible. The adhesive of the adhesive side strap 140 should be capable of maintaining the adhesive property when wet given the possibility of blood or liquid medications being in contact with the vein access needle guide assembly 100 when in use. The adhesive may be contained on the adhesive side strap 140 itself or the adhesive may be applied to both the adhesive side strap 140 and guide slide assembly 110 in the same step when producing the vein access needle guide assembly 100. Such difference may be seen in FIGS. 3 and 4 wherein the adhesive layer 150 is either a longer strip layer below the adhesive side strap 140 and guide slide assembly 110 while above the paper layer 160, as shown in FIG. 3. In another embodiment the adhesive is broken into portions and located only below the guide slide assembly 110, as the adhesive side strap 140 is provided with a separate adhesive layer prior to assembly of the vein access needle guide assembly 100. In certain embodiments the adhesive layer 150 may have an opening or aperture which corresponds with the aperture 125 of the guide slide assembly 110, or may be smaller or may be larger than the aperture 125, or even have a multiple of openings. In one embodiment the opening or aperture in the adhesive layer 150 is of a size such that the convex portion opening is unencumbered (La, is afforded clear access) through the adhesive layer 150 so as to permit entry of the needle tip for reaching the skin. In yet another embodiment the adhesive layer 150 may be a solid layer below the aperture 125. In an alternate embodiment the adhesive layer 150 may be as shown in FIG. 4.

The adhesive side strap 140 includes a layer of adhesive on the bottom to adhere to the patient's skin when in use. The adhesive may be acrylic, polymeric or any natural or synthetic adhesive capable of adhering to a patient's skin when in use and either re-adhering or capable of being removed from the patients skin with some force but not enough to remove the skin layer of the patient. Any combinations of the adhesives named and those known in the art may be used. In general any retaining means may be employed which is capable of retaining the needle guide assembly to the skin of the patient while in use.

The vein access needle guide assembly 100 may be produced in a plurality of sizes, with different sized guide slide assemblies 110 to accommodate the different standard needle gauges used by medical professionals. One example is a rectangular vein access needle guide assembly with dimensions of about 5 mm to about 50 mm wide and about 10 mm to about 100 mm long with the concave portion 120 and convex portion 130 forming an aperture 125 with a diameter of about 0.5 mm to about 10 mm. These dimensions may be changed depending on the patient's size and the size of the needle. For example, the size may be changed dependent on the target patient, such as children compared to adults. In one example the dimensions are about 10 mm to about 30 mm wide and about 45 mm to about 75 mm long with the concave portion 120 and convex portion 130 forming an aperture 125 with a diameter of about 1.0 mm to about 3.5 mm.

The angle of the guide slide 110 is to be configured to appropriately guide the needle into the vein without causing extravasation. Those appropriately trained in the art will appreciate that if a needle is inserted at the wrong angle or too far within a vein that it will damage the vein wall, causing discomfort, bruising, and even hematoma.

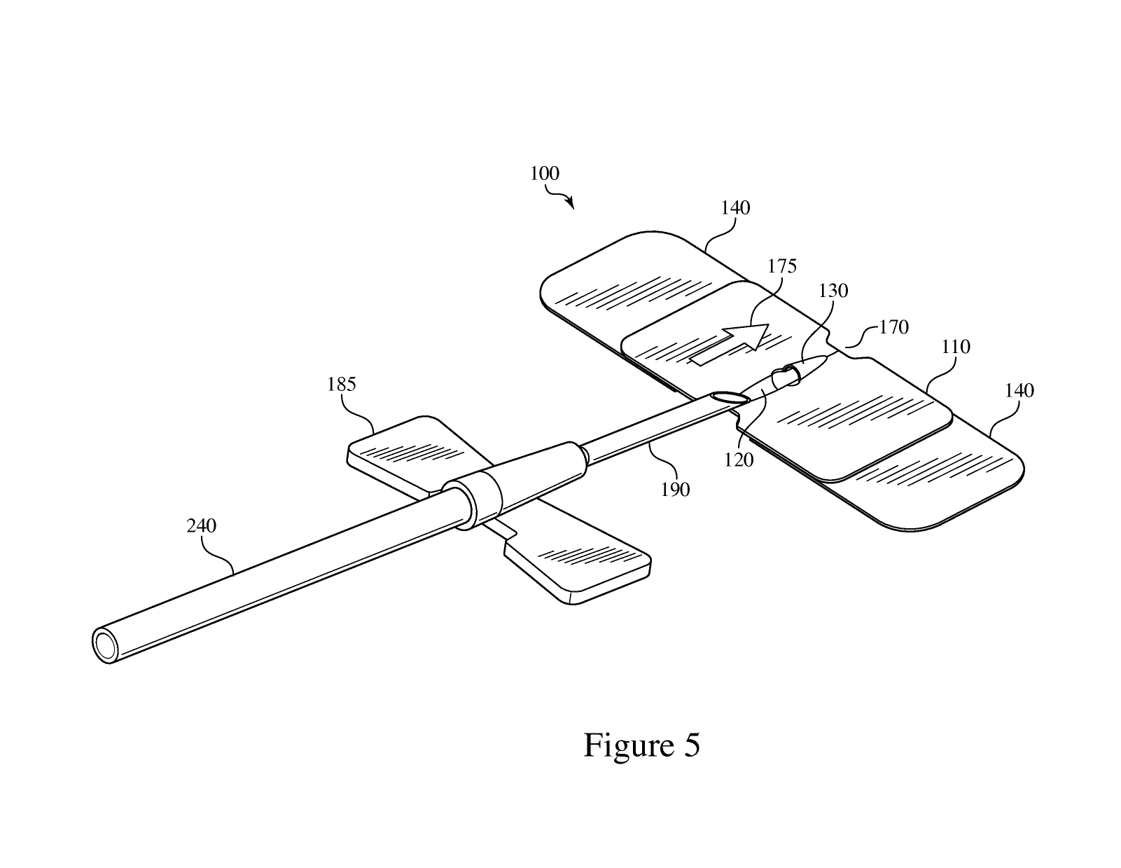

FIG. 2 shows the guide slide assembly 110 in more detail. The guide slide assembly 110 has a concave portion 120 and convex portion 130 forming an aperture 125. The aperture 125 may be defined only by the edges of each of the concave portion 120 and convex portion 130 or it may be larger when viewed from a top view. The guide slide assembly 110 may optionally include one or more guide notches 170. In a further embodiment shown in FIG. 5 the guide slide assembly 110 may include a directional indicator 175, which may be a graphic, such as an arrow pointing in the direction for the user to insert the needle into the vein access guide slide assembly 100. The direction indicator 175 could be comprised of words such as “INSERT HERE” or “UP” or any combination of graphics and/or words.

In an alternate embodiment of the guide slide assembly 110 only a concave portion 120 is included to guide the needle into the patient's skin when in use. In this embodiment the remaining portion of the guide slide assembly 110 is flat and flush with the surface of the patient's skin. Again, an optional graphic may be included. In yet another embodiment of the guide slide assembly 110 has a concave portion 120 which is formed to extend beneath the underside of the guide slide assembly 110. In a further embodiment the concave portion 120 may be formed as a depression in the guide slide assembly 110. Other combinations are possible.

Reference character 110 of FIGS. 3 and 4 is a vein access needle guide is assembly with adhesive-backed side straps 140 shown; such an assembly would further allow the vein access needle guide assembly 100 to affix securely atop the patient's skin without slipping off. FIG. 3 shows one embodiment of the vein access needle guide assembly 100 with the guide slide assembly 110 in a position on top of the adhesive-backed side straps 140 while FIG. 4 is a different embodiment of the vein access needle guide assembly 100 wherein the guide slide assembly 110 is in a position with the edges of one distal end affixed below a distal end of the adhesive-backed side straps 140.

FIG. 3 is an exploded view of one embodiment of the vein access needle guide assembly 100 with the different parts shown. The vein accessible needle guide assembly 100 with two side straps 140 to be affixed on each side of a distal end of the guide slide assembly 110 is shown with an adhesive layer 150 as well as a paper liner layer 160 that protects the adhesive portion until use. It may be preferable that the paper liner layer 160 have off-set peel, tabs (not shown).

FIG. 5 shows a needle entering into the vein access needle guide with side straps 110 that are affixed atop of a patient's skin. It can be noted that the needle slide 110 is aligned atop of the vein so that when a needle is inserted it will enter the cannula of the vein.

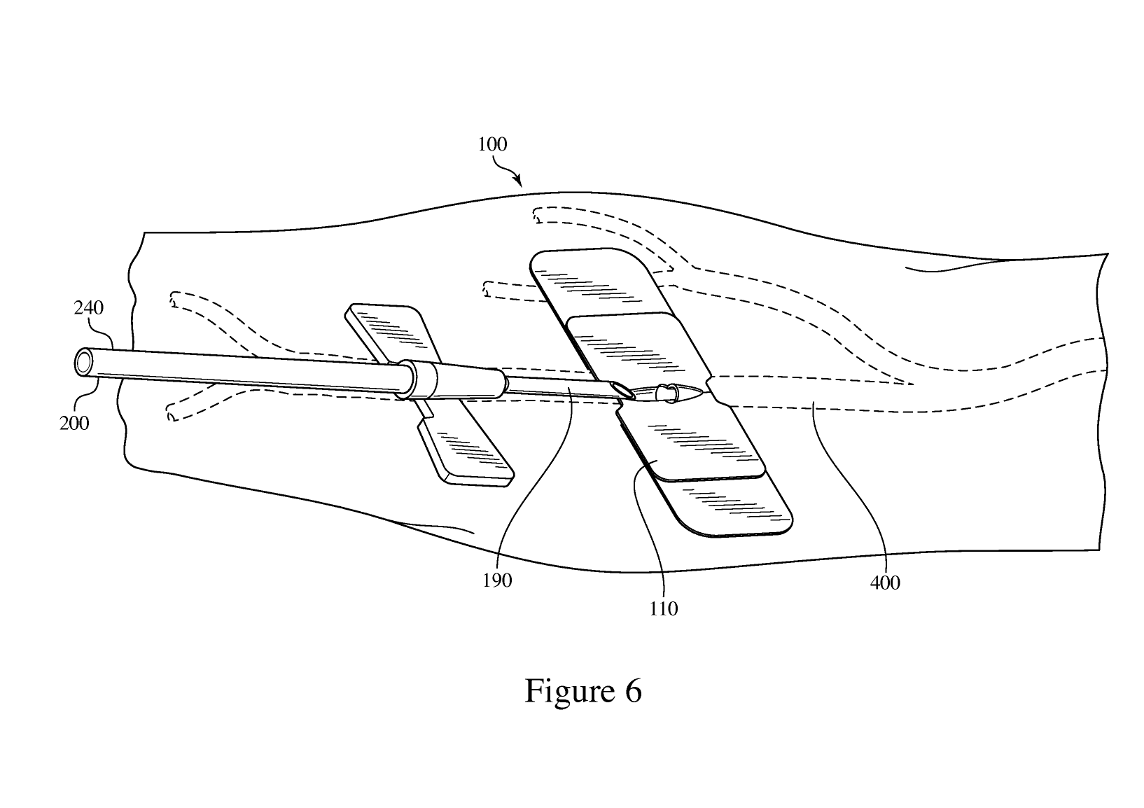

A needle is also shown in FIG. 5 where the needle tip 190 is on the edge of the concave portion 120 of the guide slide assembly 110 but not within the aperture 125. The needle may be any conventional needle having a needle tip 190 of various diameters capable of insertion into the aperture 125. The needle shown in FIGS. 5 and 6 is one example of a needle and in this embodiment is a winged-tip butterfly needle including a winged stabilizer 185. Some examples of needle tip 190 diameters are about 1.8 mm to about 22 mm These dimensions may change depending on the size of the needle. In use, the needle is moved in a direction to correspond to the flow of blood in the target vein, and in this instance is moved such that the needle tip 190 is aligned with the concave portion 120 of the guide slide assembly 110 and moves into the aperture 125 and is inserted into the patient's skin, with the convex portion 130 and the entire guide slide assembly 110 and the vein access needle guide assembly 100 securing the needle while in use.

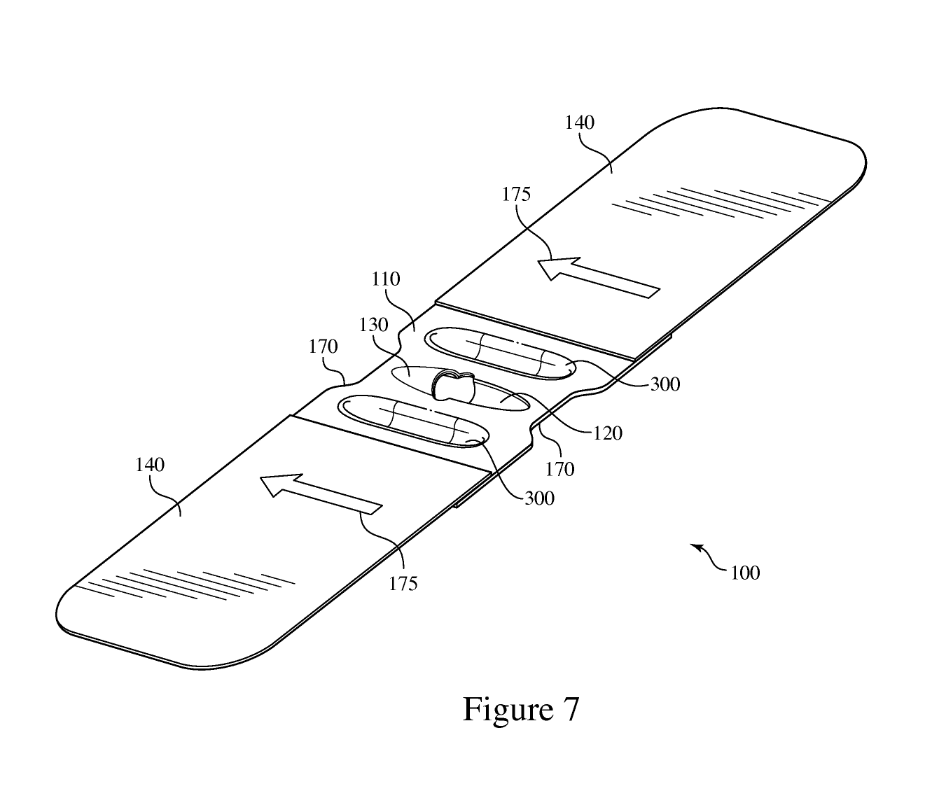

Another embodiment of the present invention is shown in FIG. 7 wherein the guide slide assembly 110 of the vein access needle guide assembly 100 includes a pair of stabilization means. The stabilization means are a concave indentation 300, in this embodiment an oval shaped concave indentation 300 is parallel to the concave portion 120, convex portion 130, and aperture 125. It is preferred that there be a pair of stabilization means 300, one located on each side of the concave portion 120, convex portion 130, and aperture 125. The stabilization means 300 acts to stabilize the target vein so that it is less likely to roll or move away from the needle, escaping puncture during the venipuncture procedure. To prevent rolling, firm pressure is applied below and to the side of the vein to stabilize it as the needle is inserted. The stabilization means 300 may so act as a stabilizer of the target vein during the venipuncture procedure. It is envisioned that the stabilization means 300 could also be a series of smaller round concave indentations or other known shapes and sizes.

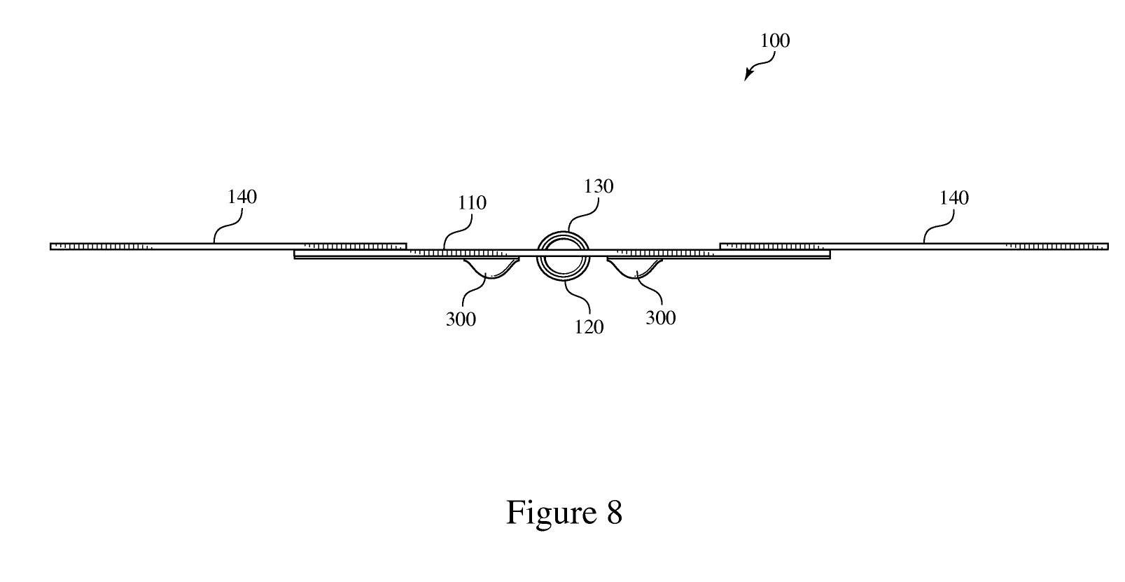

FIG. 8 is a side view of the embodiment of the present invention as shown in FIG. 7. The concave and convex features of the guide slide assembly 110 are shown including the convex portion 130, the concave portion 120, and the pair of stabilization means 300.

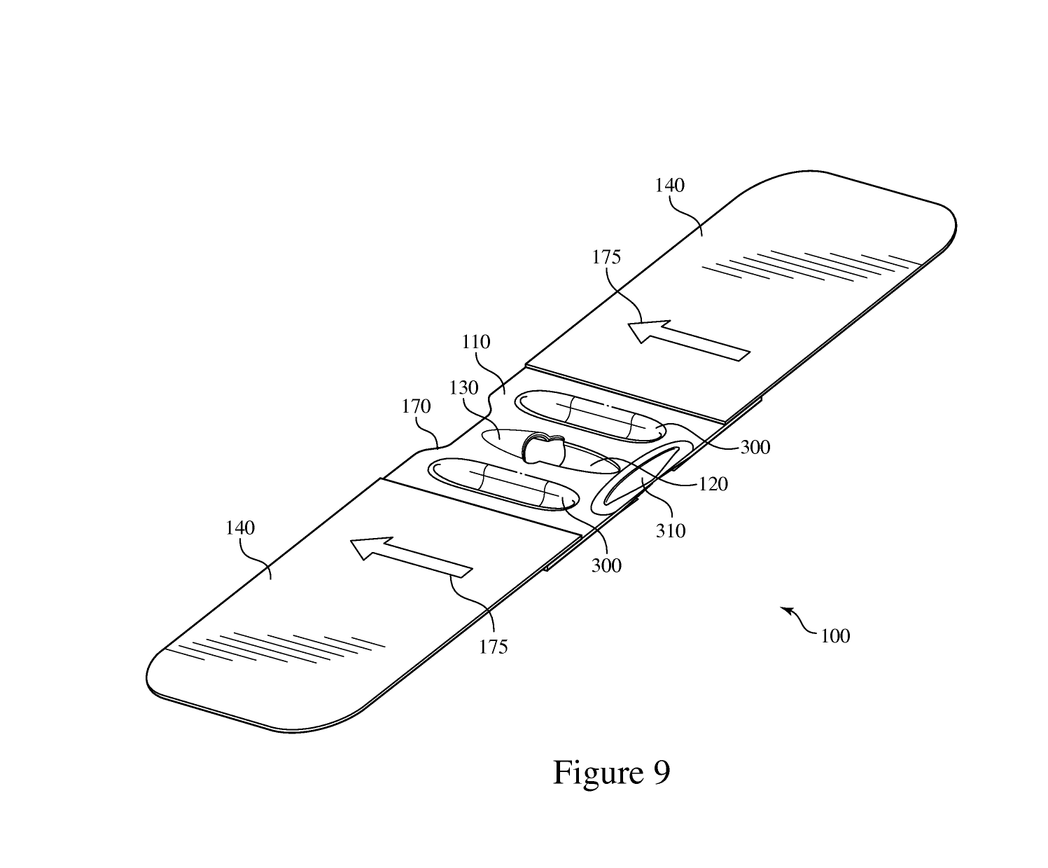

Yet another embodiment of the present invention is shown in FIG. 9 wherein the guide slide assembly 110 of the vein access needle guide assembly 100 includes a pair of stabilization means 300 and an angled support 310. The angled support 310 is convex and is preferably angled so as that the inserted needle has an optimum angle for puncturing the patient's skin and vein, while not passing through the vein's lower wall and at the same time encouraging free flow of the blood for collection during venipuncture. The angle of the angled support 310 may be between about 5° to about 90°. The angled support 310 may be in the shape of a bump, oval, triangle, or any other shape which is capable of supporting a needle during venipuncture. The angled support 310 could be a shape such that the middle has a notch or indentation (such as the shape of a “U” when viewed from the side) to better secure the needle during venipuncture.

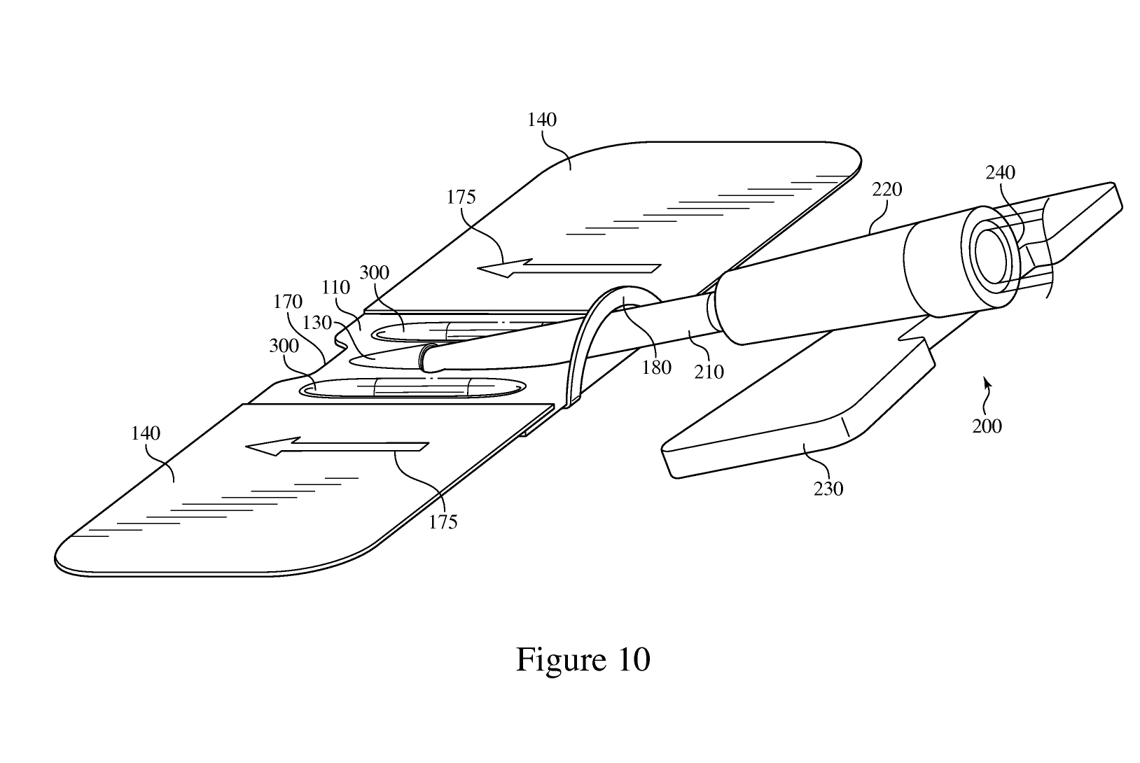

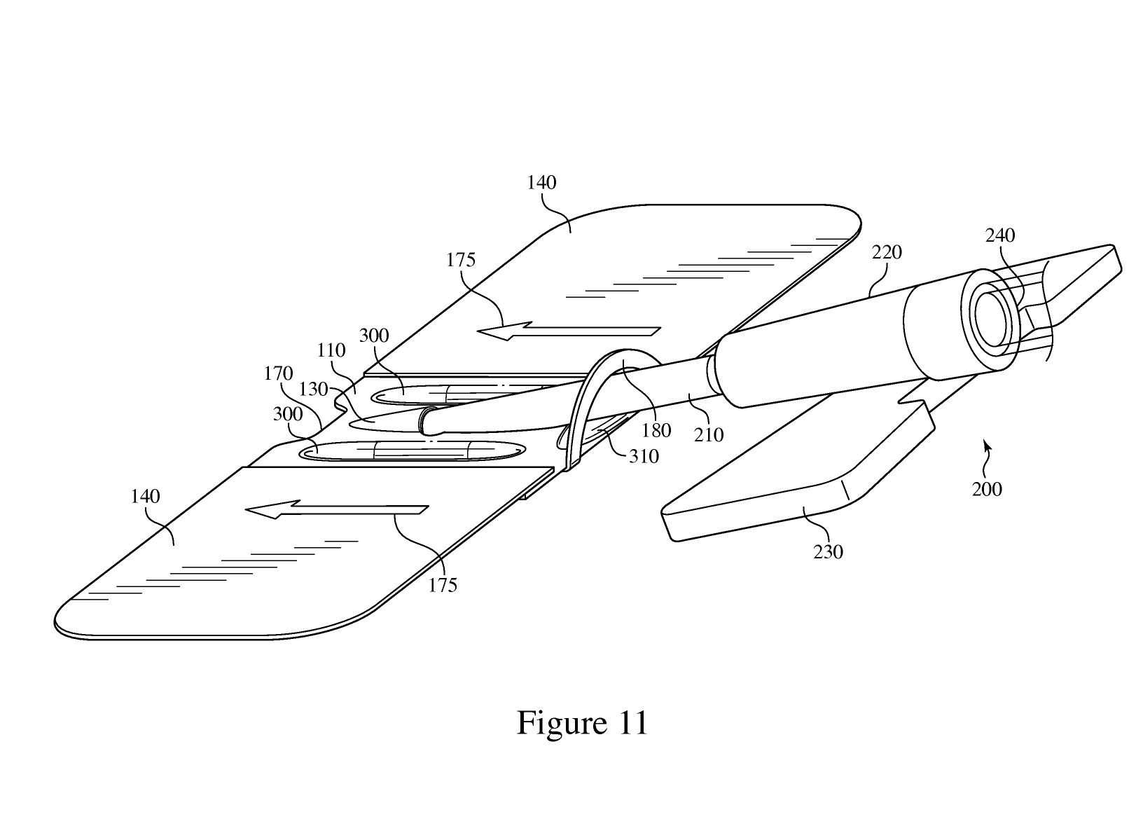

To better secure the needle during venipuncture a tab 180 is shown in FIGS. 10 and 11. The shape of the tab 180 is attached to one end of the guide slide assembly 110 nearest to the concave portion 120 which is the location of the needle during use in venipuncture. The tab 180 defines an opening for insertion of the needle during venipuncture and the diameter of the tab 180 may vary depending on the diameter of the needle being inserted during venipuncture. The tab 180 may be any shape with the shape shown in FIG. 10 as a semi-circle. It is envisioned that the tab 180 could be in the shape of a square, rectangle, triangle, or any other shape with an opening for the insertion of a needle during venipuncture. The tab 180 is especially useful when the guide slide assembly 110 includes an angled support 310 so that the inserted needle resting on, the angled support 310 is further secured by the tab 180 above the needle. The tab 180 may be made out of a rigid or semi-rigid material and could be translucent or transparent. The tab 180 may be comprised of a polymer film, plastic, nonwoven or woven material, or the material may be combinations thereof.

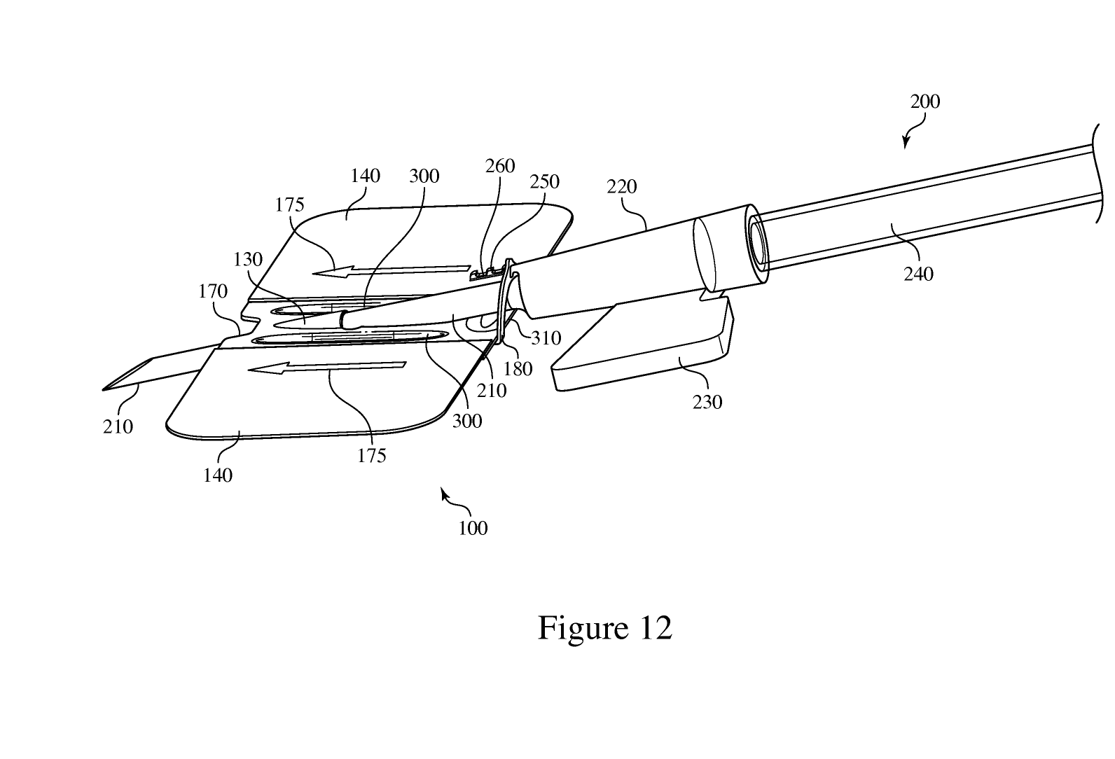

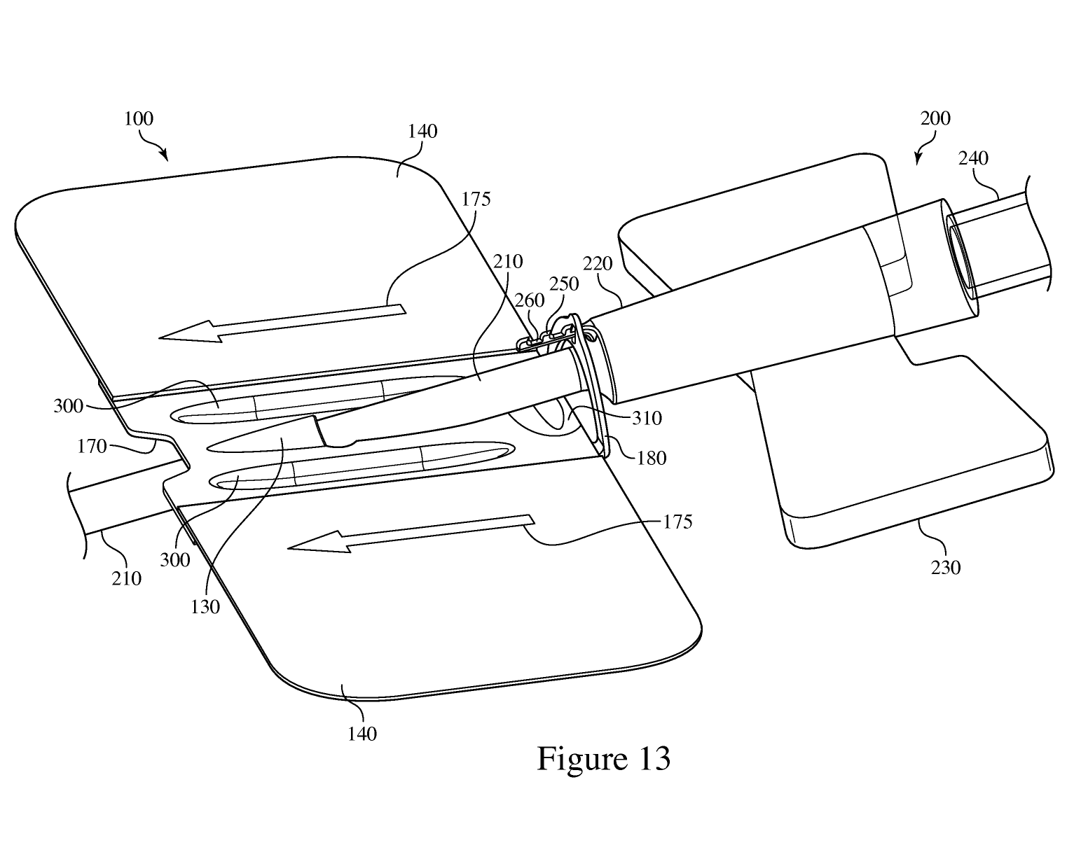

A further safety feature includes a locking means. In one embodiment the locking means includes a set of teeth-like features on the end of a needle hub 220, as shown in FIGS. 12 and 13. The needle 200 is shown already inserted into the tab 180 and the aperture 125 (not shown) and is supported by the convex portion 130 the angled support 310 and the tab 180. In this embodiment, as shown in more detail in FIG. 13, the upper end of the tab 180 includes an aperture through which the teeth 250, 260 are inserted with the teeth extending in an upward direction in this embodiment, though the opposite direction is also possible. Once the teeth 250, 260 are inserted in this embodiment the tab 180 rests within the lower portion of the teeth 260 and the upper portion of the teeth 250 locks against the tab 180. A series of teeth 250, 260 are included so that the needle hub 220, when moved in a downward direction as the needle 200 is inserted into the vein access needle guide assembly 100, moves in a direction such that the teeth 250, 260 pass in one direction and cannot be retracted out of the aperture in the tab 180. Thus the needle 200 is locked into the inventive vein access needle guide assembly 200. The locking means, for example the teeth 250, 260, may be comprised of the same or not the same material as the tab 180. The locking means, for example the teeth 250, 260, may be made out of a rigid or semi-rigid material and could be translucent or transparent. The locking means, for example the teeth 250, 260, may be comprised of a polymer film, plastic, nonwoven or woven material, or the material may be combinations thereof.

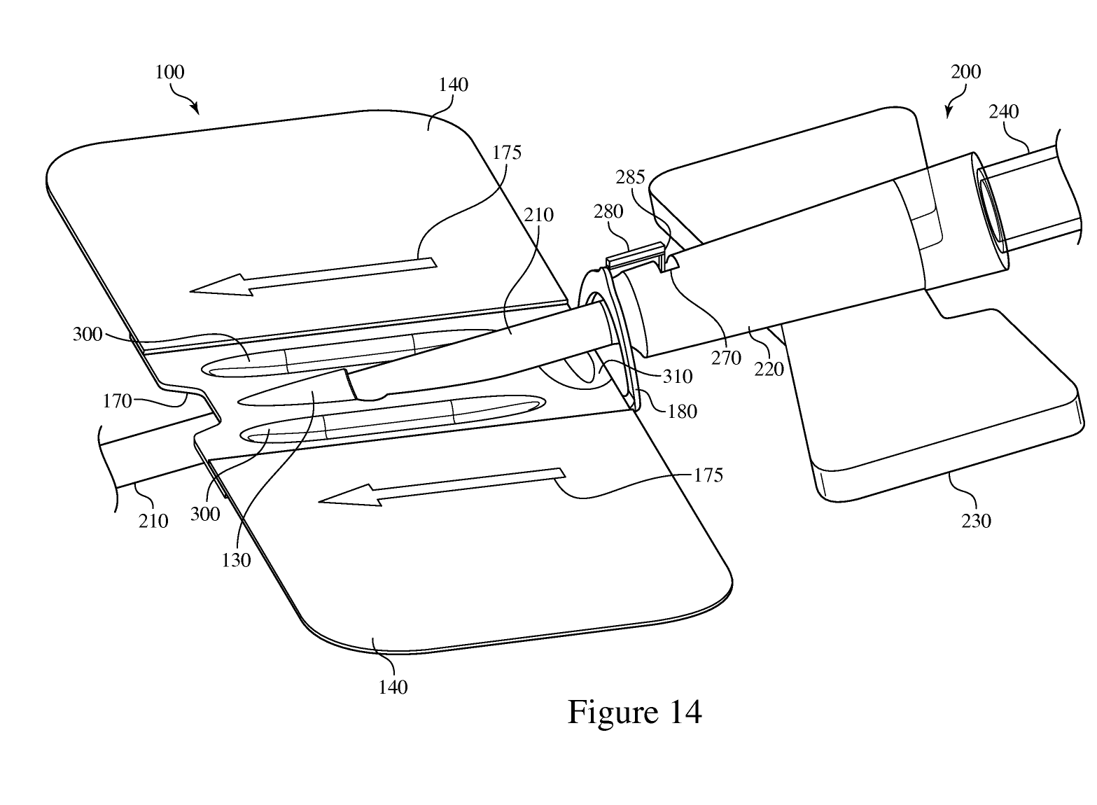

Another embodiment of the invention and of another locking means is shown in FIG. 14 wherein the tab 180 includes a horizontal member 280 which on its distal end has a downward vertical member 285 which can rest or lock within an aperture 270 in the needle hub 220. The needle 200 is shown already inserted into the tab 180 and the aperture 125 (not shown) and is supported by the convex portion 130, the angled support 310 and the tab 180. In this embodiment the upper end of the tab 180 includes a horizontal member 280 which is located on the opposite side of the tab extending away from the guide slide assembly 110. The horizontal member 280 has a distal end including a downward vertical member 285 which either rests on a needle hub 220 or fits into and locks within an aperture 270 on the top of a needle hub 220. Once the vertical portion of the horizontal member 285 is inserted into the aperture 270 it cannot be retracted out easily and thus the needle 200 cannot be retracted out easily. Thus the needle 200 is secured and/or locked into the inventive vein access needle guide assembly 200. The horizontal member 280 may be comprised of the same or not the same material as the tab 180. The locking means, for example the horizontal member 280 may be made out of a rigid or semi-rigid material and could be translucent or transparent. The locking means, for example the horizontal member 280 may be comprised of a polymer film, plastic, nonwoven or woven material, or the material may be combinations thereof. Other locking means may be employed and the two embodiments herein are mere examples and not limiting to the actual locking means or mechanisms used in each iteration of the inventive vein access needle guide assembly 100.

It is envisioned that the vein access needle guide assembly 100 would be packaged in a sterilized package, either as a stand-alone product or in combination with multiple needle guides or other products such as a needle. Other kits including the vein access needle guide assembly 100 could include a cleansing agent in a sterile package, a tourniquet (or a cuff as disclosed in co-pending Provisional Application 62/207,088 entitled “Vein Identifier Assembly And Methods Of Use”), the vein access needle guide assembly 100, a needle (including a needle having a needle hub 220 with either an aperture 270 or a locking means 250, 260), a piece of sterile gauze and a sterile bandage.

A method of venipuncture using the inventive vein access guide assembly 100 may include a number of steps. First, the target vein is chosen (if an AV fistula is not the target location) such as on the outside of the forearm, on the back of the hand, in the antecubital fossa or any other body location. Next, the skin on the patient located where the user wants to access the vein is cleaned by a cleansing agent. The cleansing agent used to prepare the insertion site may be iodine, povidone-iodine, or ethyl alcohol. The cleansing agent may be an alcohol swab, preferably 70% isopropyl alcohol. The vein is palpated, and to dilate the vein a tourniquet may be wrapped around the arm proximal to the intended site of puncture. Optionally a tourniquet or other constricting means may be placed at a location near the target vein so that the target vein protrudes or is more easily visualized or subject to tactile manipulation and touch. The patient may be asked to pump his or her fist if the target vein is located in the arm.

Next, if the vein access needle guide 100 is packaged in a sterile packaging, then packaging is opened. The vein access guide assembly 100 is aligned with the target vein such that, the concave portion 120 is aligned with the flow of the blood in the target vein as the inserted needle would also align with the blood flow. The paper liner layer 160 is peeled back exposing the adhesive layer 150 and the vein access needle guide 100 is affixed atop the patient's skin, appropriately aligning over the target vein, preferably with the needle guide slide 110 located over the target vein and if including the stabilization means 300 such stabilization means 300 is optionally located on each side of the target vein. As shown in FIGS. 6 and 10-14, a needle tip 190 is inserted into the guide slide 110 to access the vein and the concave portion 120 secures the needle tip 190. The angle of the needle tip 190 may be between about 5° to about 90°, preferably about 10° to about 40°, and more preferably about 15° to about 30°, dependent on the diameter of the needle tip and the size of the target vein 400. If the guide slide assembly includes the angled support 310 the distal end 210 of the needle 200 is supported at the chosen angle. After the skin is punctured, little resistance should be felt by the user as the needle tip 190 should pass through the subcutaneous tissue, but a sudden slight resistance may be felt as the needle tip 190 hits the wall of the vein. At this point the needle tip 190 may be cautiously advanced, with the needle 200 held nearly flush with the skin as the needle 200 is in the concave portion 120 of the guide slide assembly 110 and secured by the convex portion 130. Slight upward pressure applied by the user and the guide slide assembly 110 itself may aid in keeping the needle tip 190 in the target vein 200 as the needle tip 190 is advanced into the lumen of the target vein 200. A successful venipuncture results in blood flowing back into the hub 220 of the needle 200 or into the catheter or vacuum tube (240) attached to the distal end 210 of the needle 200, and the needle tip 190 usually can be felt to be in the vein. If these signs are absent, the needle tip 190 is not in the target vein 400, in which case it is usually best to remove the needle 200, apply pressure to the puncture site, and start the procedure again, using new equipment.

If the user has an alternate embodiment of the inventive vein access needle guide assembly 100 including an angled support 310, the tab 180, and/or locking means (either the combination of 250, 260 or the combination of 270, 280, 285 or any other locking means), then the vein access guide assembly 100 is aligned with the target vein such that the concave portion 120 is aligned with the flow of the blood in the target vein as the inserted needle would also align with the blood flow. The paper liner layer 160 is peeled back exposing the adhesive layer 150 and the vein access needle guide 100 is affixed atop the patient's skin, appropriately aligning over the target vein, preferably with the needle guide slide 110 located over the target vein and each of the pair of stabilization means 300 located on each side of the target vein. A needle tip 190 is inserted into the opening of the tab 180 and through into the aperture 125 of the guide slide 110 to access the vein with the distal end 210 of the needle 200 resting on the convex portion 130 with the concave portion 120 and tab 180 securing the needle 200. If the guide slide assembly includes the angled support 310 the distal end 210 of the needle 200 is supported at the chosen angle. After the skin is punctured, little resistance should be felt by the user as the needle tip 190 should pass through the subcutaneous tissue, but a sudden slight resistance may be felt as the needle tip 190 hits the wall of the vein. At this point the needle tip 190 may be cautiously advanced, with the needle 200 held nearly flush with the skin as the needle 200 is in the concave portion 120 of the guide slide assembly 110 and secured by the convex portion 130 and the tab 180. Slight upward pressure applied by the user and the guide slide assembly 110 itself may aid in keeping the needle tip 190 in the target vein 200 as the needle tip 190 is advanced into the lumen of the target vein 200. If the embodiment of the vein access needle guide assembly 100 includes a locking means such means may be activated so that the needle hub 220 is secured or locked with the tab 180. For instance, if the locking means is a series of teeth 250, 260 located on a distal end of the needle hub, such teeth 250, 260 are inserted into an aperture located on the top potion of the tab 180 and when the needle tip 190 is located within the target vein at the chosen angle and depth, the needle 200 is locked to the inventive vein access needle guide assembly 100 as the bottom of the aperture rests within the horizontal portion 260 of the tooth and the upper portion of the aperture rests against the vertical portion 250 of the tooth. Thus the needle 200 is locked into the inventive vein access needle guide assembly 100. If in the alternate, the locking means includes an aperture 270 in the upper distal end of the needle hub 220 into which a vertical portion 285 of a horizontal member 280 on the tab 180 extending away from the guide slide assembly 110, such locking means is activated when the needle tip 190 is inserted into the opening of the tab 180 and the aperture 125 of the guide slide assembly 110 and the vertical member 285 slides against the upper side of the needle hub 220 until it falls within the aperture 270 such that the entire needle 200 cannot be retracted out of the inventive vein access needle guide assembly. A successful venipuncture results in blood flowing back into the hub 220 of the needle 200 or into the catheter or vacuum tube 240 attached to the distal end 210 of the needle 200, and the needle tip 190 usually can be felt to be in the vein.

In a successful venipuncture using the inventive vein access needle guide assembly 100, the user now has access to the patient's vein and blood may be drawn and fluids or medication put into the circulatory system. At a later time the needle 200 is removed from the patient's vein by the user, the vein access needle guide assembly 100 is removed, and optionally a sterile piece of gauze, and a conventional adhesive bandage is placed atop the location on the patient's skin where the vein was accessed to provide pressure until the local bleeding stops.

The inventive method may be used to obtain blood for diagnostic purposes, to monitor levels of blood components, to administer therapeutic treatments such as medications (e.g., intravenous antibiotics), nutrition, or chemotherapy for cancer patients. Other uses of the inventive venipuncture method and other uses for inventive vein access needle guide assembly 100 include removing blood due to excess levels of iron (e.g., chelation therapy) or erythrocytes (Le., red blood cells) or to collect blood for later uses such as donor blood and transfusions. Other uses may be contemplated if relating to insertion of a needle into a patient's skin to access a vein.

The inventive vein access needle guide assembly 100 assists the user in the proper insertion of a needle into a vein. The inventive vein access needle guide assembly 100 is portable and does not impinge on the patient's skin but rather is easily placed on the patient's skin in the location of the target vein and is retained on the skin sufficiently for the venipuncture and securing of the needle without being so secure that the user cannot remove the vein access needle guide assembly 100 easily with minimal force.

Further, the vein access needle guide assembly 100 of the present invention stabilizes the target vein during venipuncture and may reduce the occurrence of a “rolling” vein during venipuncture. The vein access needle guide assembly 100 of the present invention supports the inserted needle at a certain angle to provide improved venipuncture and collection of a blood sample. The vein access needle guide assembly 100 of the present invention provides a more secure venipuncture based on the support, securing and possible locking of the needle to the inventive vein access needle guide assembly during the procedure resulting in less movement of the needle during the procedure and potentially less pain and injury to the patient and potentially quicker procedure of the actual blood draw. The vein access needle guide assembly 100 of the present invention may lessen anxiety and pain for the patient in that that vein access needle guide assembly 100 is small and pliable while also assisting in the venipuncture which results in a quicker venipuncture process, as well as a more accurate process. Further, the user may experience less anxiety also based on the needle guide slide 710 of the inventive vein access needle guide assembly 700 and being able to easily access the target vein and not having to rely on the user's own visual cues or tactile impressions such that the risk of puncture of the user's finger is lessened or even totally removed.

Further, the inventive method and inventive vein access needle guide assembly 700 may be used in venipuncture related to a fistula, including an AV fistula. The patient may therefore experience less pain and the user experience a quicker and/or easier and/or more accurate and less stressful, venipuncture of the skin surrounding the AV fistula.

The inventive method and inventive vein access needle guide assembly 100 may be cheaper and more easily available than current vein guide apparatuses and thus may be used by hospitals, clinics and even small individual doctor's offices.

The invention has been described in terms of embodiments thereof, but is more broadly applicable as will be understood by those skilled in the art. The scope of the invention is only limited by the following claims.

1. A needle guide assembly for vein access, comprising
a guide having (a) a convex portion, (b) a concave portion, (c) an aperture and (d) stabilization means for stabilizing a vein against moving away and escape puncture, the concave portion and the convex portion meeting each other to define the aperture between the concave portion and the convex portion, each of the convex portion and the concave portion extending away from the aperture in opposite directions from each other in a tapering manner and terminating the tapering at respective locations away from a peripheral edge of the guide, the convex portion being made of a transparent or translucent material, the stabilization means being a pair of elongated indentations that extend substantially parallel to each other and between which are the convex portion, the concave portion and the aperture; and
retaining means for retaining the guide to skin.

2. The assembly of claim 1, characterized in that the indentations project from an underside of the guide and have convex curvature from the underside.

3. The assembly of claim 1 further characterized by a kit that contains a sterile package enclosing the guide, the means for retaining the guide to skin, a packaged sterile alcohol swab, a packaged sterile needle and a packaged sterile bandage.

4. The assembly of claim 1 characterized in that the convex portion extends from a topside of the guide, the concave portion extends from an underside of the guide.

5. The assembly of claim 1, characterized in that the retaining means includes an adhesive layer having an opening in alignment with at least one of the concave and convex portions.

6. The assembly of claim 5, characterized in that the concave portion fits into the opening in the adhesive layer.

7. The assembly of claim 1 further characterized by a tab along the peripheral edge of the guide in a shape and diameter capable of housing a needle.

8. The assembly of claim 1 further characterized by a convex portion along the peripheral edge of the guide at an angle of about 5° to about 90°.

9. The assembly of claim 1 further characterized by a locking means to lock a hub of the needle to the upper portion of the tab.

10. The assembly of claim 9 wherein the locking means include teeth on a distal end of a needle hub inserted into an aperture located on the top potion of the tab.

11. The assembly of claim 9, wherein the locking means include an aperture in an upper distal end of a needle hub and a vertical portion of a horizontal member on the tab extending away from a guide slide assembly, the locking means being activated as a needle tip is inserted into the opening of the tab and the aperture of the guide slide assembly and the vertical member slides against the upper side of the needle hub until falling within the aperture such that an entirety of the needle cannot be retracted out.

12. The assembly of claim 1, characterized in that the guide has two sets of pairs of opposite edges that form the periphery, the convex portion and the concave portion each being symmetric relative to a line of symmetry, further comprising two side straps adhered to the guide and extending outward past one of the sets of pairs of the opposite edges, each of the two side straps having a respective edge spaced away from each other that are substantially equidistant from the line of symmetry.

13. The assembly of claim 1, characterized in that the guide has two sets of pairs of opposite edges that form the periphery, the opposite edges of one of pairs of the two sets each defining a respective notch so as to provide for two notches, the concave portion and the convex portion extending from the aperture away from each other between the two notches so that the convex portion tapers in a direction toward one of the two notches and the concave portion tapers in a direction toward a remaining one of the two notches.

14. The assembly of claim 1, characterized in that the guide has a two sets of pairs of opposite edges that form the periphery, at least one of the opposite edges of one of the pairs of the two sets defining a notch, at least one of the convex portion and the concave portion tapering in a direction toward the notch.

15. The assembly of claim 8, characterized by said retaining means including an adhesive layer that has a further notch in alignment with the notch of the guide and being substantially identical in shape to that of the notch of the guide.

16. The assembly of claim 1, characterized in that the guide has a direction indicator marked on a topside of the guide that points in a same direction that the convex portion tapers.

17. The assembly of claim 1, characterized by a needle stabilizer having a channel that extends in a channel direction to accommodate insertion of a needle, spacing the needle stabilizer from the guide by a distance sufficient to enable simultaneous insertion of the needle through both the channel of the needle stabilizer and the aperture of the guide in alignment so that a tip of needle enters the convex portion of the guide as a portion of a remainder of the needle is within the channel.

18. The assembly of claim 1, characterized in that the retaining means includes an adhesive layer, that adhesive layer having an adhering side, further characterized by a paper layer against the adhering side of the adhesive layer and peelable under manual force for removal from the adhering side.

19. The assembly of claim 1, characterized in that the convex portion extends from a topside of the guide, the topside of the guide having a depression forming the concave portion.

20. A method of assembling a needle guide assembly for vein access, comprising
providing a guide having, (i) a convex portion, (ii) a concave portion, (iii) an aperture and (iv) a stabilization means for stabilizing a vein against moving away and escape puncture, the concave portion and the convex portion meeting each other to define the aperture between the concave portion and the convex portion, each of the convex portion and the concave portion extending away from the aperture in opposite directions from each other in a tapering manner and terminating the tapering at respective locations away from a peripheral edge of the guide, the convex portion being made of a transparent or translucent material, the stabilization means being a pair of elongated indentations that extend substantially parallel to each other and between which are the convex portion, the concave portion and the aperture; and
retaining the guide to skin.

21. The method of claim 20, further characterized by the steps of:
(a) locating a vein on a skin surface for insertion of a needle;
(b) cleansing an area of the skin surface in relation to the vein chosen for insertion of the needle;
(c) palpating the vein;
(d) adhering the guide onto the skin in relation to the chosen vein aligning the stabilization means along the chosen vein to effect the retaining of the guide to the skin;
(e) inserting a distal needle tip of the needle along the concave portion of the guide through the aperture and to reach the skin surface: and
(f) securing the needle under the convex portion of the guide.

22. The method of claim 21, further characterized by the steps of:
(g) attaching a collection container to a proximal end of the needle to receive drawn blood from the vein;
(h) removing the distal needle tip away from the skin surface;
(i) removing the guide from the skin surface;
(j) placing a sterile piece of gauze with pressure on he skin surface where the distal needle tip was removed; and
(k) placing a sterile bandage on the skin surface where the distal needle tip was removed.

23. The method claim 21, further characterized by the steps of:
(g) attaching a proximal portion of the needle to a container containing medicine;
(h) discharging the medicine through the needle to pass through the distal needle tip;
(i) removing the distal needle tip from the skin surface;
(j) removing the guide from the skin surface;
(k) placing a sterile piece of gauze with pressure on the skin surface where the distal needle tip was removed; and
(l) placing a sterile bandage on the skin surface where the distal needle tip was removed.

24. The method of claim 21, further characterized by the inserting the distal needle tip step including the step of inserting the distal needle tip of the needle within an opening in a tab portion along a peripheral edge of the guide and inserting within the concave portion of the guide through the aperture and to reach the skin surface.

25. The method of claim 21, further characterized by the inserting the distal needle tip step including the step of supporting the needle on a convex portion of the guide along a peripheral edge of the guide and inserting within the concave portion of the guide through the aperture and to reach the skin surface.

26. The method of claim 24, further characterized by the step of locking an upper portion of the tab of the guide to a hub of the needle via a locking means.

27. The method of claims 26 wherein the locking means includes an aperture in an upper distal end of a needle hub and a vertical portion of a horizontal member on the tab extending away from a guide slide assembly, the locking means being activated as a needle tip is inserted into the opening of the tab and the aperture of the guide slide assembly and the vertical member slides against the upper side of the needle hub until falling within the aperture such that an entirety of the needle cannot be retracted out.

28. The method of claim 20, characterized in that the guide has two sets of pairs of opposite edges that form the periphery, the convex portion and the concave portion each being symmetric relative to a line of symmetry, further comprising adhering two side straps to the guide and extending the two side straps outward past one of the sets of pairs of the opposite edges, each of the two side straps having a respective edge spaced away from each other that are substantially equidistant from the line of symmetry.

29. The method of claim 20, characterized in that the guide has two sets of pairs of opposite edges that form the periphery, the opposite edges of one of pairs of the two sets each defining a respective notch so as to provide for two notches, the concave portion and the convex portion extending from the aperture away from each other between the two notches so that the convex portion tapers in a direction toward one of the two notches and the concave portion tapers in a direction toward a remaining one of the two notches.

30. The method of claim 20, characterized in that the guide has two sets of pairs of opposite edges that form the periphery, at least one of the opposite edges of one of the pairs of the two sets defining a notch, at least one of the convex portion and the concave portion tapering in a direction toward the notch.

31. The method of claim 20, characterized by a needle stabilizer having a channel that extends in a channel direction to accommodate insertion of a needle, spacing the needle stabilizer from the guide by a distance sufficient to enable simultaneous insertion of the needle through both the channel of the needle stabilizer and the aperture of the guide in alignment so that a tip of needle enters the convex portion of the guide as a portion of a remainder of the needle is within the channel.
